(19) United States
(12) Patent Application Publication
(10) Pub. No.: US 2021/0057073 A1
(43) Pub. Date: Feb. 25, 2021

(54) MIXER AND DISPENSER FOR LIQUID MEDICINE

(71) Applicant: RYAH Medtech, Inc., Brooklyn, NY (US)

(72) Inventor: Boris Goldstein, New York, NY (US)

(21) Appl. No.: 17/001,926

(22) Filed: Aug. 25, 2020

Related U.S. Application Data
(60) Provisional application No. 62/891,376, filed on Aug. 25, 2019.

(51) Int. Cl. G16H 20/13; G06K 19/06; G06K 19/07; G16H 10/40

(52) U.S. Cl. CPC G16H 20/13; G16H 10/40; G06K 19/0723; G06K 19/06037

(57) ABSTRACT

This invention concerns a dispenser of liquid medicine. The dispenser has multiple cartridges containing separate liquids in each cartridge. In operation, the cartridges dispense their respective content into a reservoir, the liquid is then mixed before being dispensed through a mouthpiece and consumed orally. The dispenser has electronic components and can be controlled by an internal computer or by an outside application or software on a mobile device or a computer. The liquid dispensed may be changed and tailored using the internal computer or outside application or software.

### BACKGROUND OF THE INVENTION

### Field of the Invention

This invention relates to a device to mix different liquids previously stored in different compartments to form a mixture with ingredients well mixed and evenly distributed throughout the mixture.

### Description of the Related Technology

Administering liquid medicine for oral consumption is a popular practice for many. Various instruments are available to facilitate this delivery, including syringes, pipettes, and droppers. All these instruments focus on accurate delivery of one medicine that is premixed to a subject in need of this particular delivery route, such as a baby or an animal, or to anyone desired.

Cannabis as medicine has evolved rapidly in recent years. Cannabis has been used for a variety of indications in many different patient populations with different needs. Together with this development is the challenge in delivering accurate dosage and finding an effective administration route.

Cannabis plants have more than one hundred cannabinoids, the active ingredients in cannabis. Popular cannabinoids include cannabidiol (CBD), cannabigerol (CBG), tetrahydrocannabinol (THC), cannabidivarin (CBDV), cannabinol (CBN), cannabichromene (CBC), among other cannabinoids. THC is known as having psychoactive effects, giving users the “high” feeling while CBD is nonpsychoactive. Each cannabinoid delivers a different therapeutic outcome and, in many cases, a mixture of cannabinoids at a certain ratio is needed for a specific indication.

Cannabis plants can exhibit wide variation in the quantity and type of cannabinoids they produce. The mixture of cannabinoids produced by a plant is known as the plant's cannabinoid profile. Selective breeding has been used to control the genetics of plants and modify the cannabinoid profile. For example, strains that are used as fiber (commonly called hemp) are bred such that they are low in psychoactive cannabinoids like THC. Strains used in medicine are often bred for high CBD content, and strains used for recreational purposes are usually bred for high THC content or for a specific chemical balance. Other breeding methods for other cannabinoids profiles are also developing. For cannabis plants bred to provide medicine, achieving a specific content of cannabinoids has become an objective that is still in the pursuing.

Cannabis medicine is delivered through many routes, including by inhalation, oral consumption, topical administration, suppository, or transdermal patches. Oral consumption is a popular way for most of the population due to the easy nature of oral consumption and relative safety with minimal side effects.

However, delivery of cannabis for oral consumption has faced many challenges, the biggest of them being dosing. Cannabis to be consumed orally is a plant extract, the dosage of which is hard to control. Where a specific mixture of cannabinoids—the active ingredients within cannabis—is desired, current technology is not available to cater for each individual's need. The ratios of cannabinoid in edible cannabis products are typically “fixed” by the plant and embedded into the consumption unit. For example, a candy with 20 mg of CBD may be available, but one with 20 mg of CBD and 10 mg CBG may not be available. Adding flavor or other components is not an option for these delivery methods.

The same challenges apply in delivering other liquid medicines. Cough syrups are available with pre-mixed flavors and measurement is required for precise dosing. Methadone, another medicine often dispensed in liquid or dissolvable tablet form, usually comes with a pre-mixed flavor that is not tasty for many. The ability to change dosage and flavors is beneficial in these circumstances.

There remains a need to deliver medicine in an orally consumable form with accurate dosing, such that the amounts of active ingredient(s) and flavor(s) may be tailored and adjusted for each user and at different times. Moreover, the need to be able to control a medicine dispensing device with precise electronic means also exists. This invention seeks to solve this problem.

### SUMMARY

This invention discloses a liquid dispenser system to mix and dispense liquid medicine from multiple cartridges contained within the system. The liquid dispenser has space for pre-made cartridges containing liquid medicines or other liquids to be assembled into. The liquid dispenser can be controlled by a remote application or software, such that a premixed, according-to-specified parameters and mixture ratios liquid medicine can be dispensed to the user by demand. The user consumes the liquid mixed and dispensed by the dispenser by sucking on a mouthpiece and swallowing the liquid dispensed or by mixing the dispensed liquid with other liquids, such as juices or coffee. Alternatively, the user can also dispense the liquid into their mouth under their tongue and hold it there to absorb the liquid sublingually and bypass metabolism.

This invention discloses a system for mixing and dispensing liquid, comprising: a reservoir configured to receive liquid from at least two sources, the reservoir having inlets capable of receiving liquid;

at least two cartridges, each of the cartridge comprises:
a dispensing means operatively connected to the cartridge to dispense liquid from the cartridge into a reservoir;
space within the cartridge to store a volume of liquid; and
an outlet at one end of the cartridge;

an electronic controller embedded within the system and configured to operate the system;

an electronic interface present on the system and configured to interact with a user to operate the system;

a power source;

a mouthpiece;

a pneumatic valve operatively connected to the reservoir and the mouthpiece; and

a housing to house the above components,

wherein each of the cartridges stores a different liquid mixture and connects physically and operatively to the reservoir at one of the inlets.

This invention provides a system as above, wherein at least one of the liquid mixtures comprises at least one cannabinoid.

This invention provides a system as above, wherein at least of one of the liquid mixtures is viscous at room temperature.

This invention provides a system as above, wherein at least one of the liquid mixtures comprises a flavor.

This invention provides a system as above, wherein the outlet is covered with a mechanical septum.

This invention provides a system as above, wherein the liquid cartridges are pre-made and replaceable.

This invention provides a system as above, wherein each of the liquid cartridges further comprises information contained in an electronic information storage means.

This invention provides a system as above, wherein the electronic information storage means is Near Field Communication or Quick Response code.

This invention provides a system as above, further comprising an activation means.

This invention provides a system as above, further comprising means to wirelessly communicate with other processors embedded within the electronic controller.

This invention provides a system as above, further comprising a computer programming product configured to wirelessly communicate with and send orders to the electronic controller.

This invention provides a system as above, wherein the computer programming product is an application or a software capable of operating on a computing article and configured to interact with a user at a user interface displayed on the computing article.

This invention provides a system as above, wherein the computing article is a mobile computing device, a desktop, a laptop, or a computer embedded into another electronic device.

This invention provides a system as above, wherein the computer programming product is further configured to collect information from the electronic information storage means present on the cartridge.

This invention provides a system as above, wherein the computer programming product is configured to control operating parameters of the system.

This invention provides a system as above, wherein the operating parameters are dispensing amount from each cartridge, mixture ratio, time of consumption, frequency of consumption, and place of consumption.

This invention provides a system as above, wherein the computer programming product is further configured to collect information on operating parameters from the system and transmit collected information to other computing articles.

This invention provides a system as above, wherein the collected information is stored on a computing article, aggregated, and analyzed to optimize the system's operation or to conduct medical research.

This invention provides a method to provide a liquid mixture to a user, comprising:

providing to a user the system as above;

assembling pre-filled cartridges into the system;

activating the dispenser using the activation means;

control operating parameters using the computer programming product on the remote computing article;

waiting for the system to prepare the liquid mixture; and

consuming the liquid mixture orally.

This invention provides a method as above, comprising the steps of:

providing the system for mixing and dispensing liquid as above to a user;

opening the computer programming product on the remote computing processor;

setting operating parameters for the system;

starting the operating procedure for the system to provide the liquid mixture for the user's consumption;

collecting data from the system operation;

transmitting collected data to a remote computing processor;

storing collected data in a computing article;

repeating the above steps with various systems and various users for a finite time;

analyzing stored data to make medical care decisions; and

aggregating data from different users and conducting research and study based on the aggregated data.

### ABBREVIATIONS

CBC: cannabichromene

CBD: cannabidiol

CBDV: cannabidivarin

CBG: cannabigerol

CBN: cannabinol

mg: milligram

mm: millimeters

ml: milliliters

LED: Light Emitting Diode

NFC: Near Field Communication

RGB: Red Green Blue

QR: Quick Response

THC: tetrahydrocannabinol

USB: Universal Serial Bus

### BRIEF DESCRIPTION OF THE DRAWINGS

FIG. 1 is the front view of the liquid dispenser with all parts assembled.

FIG. 2 is the front view of the liquid dispenser with the cap removed and the electronic interface highlighted.

FIG. 3 is a perspective view of the liquid dispenser with all parts assembled.

FIG. 4 is a side view of the liquid dispenser with the outside housing removed for viewing.

FIG. 5 shows a front view of the liquid dispenser with certain components removed such that the cartridges are visible.

FIG. 6 shows a front view of the liquid dispenser with the battery and the pneumatic valve visible.

FIG. 7 is the bottom view of the liquid dispenser.

FIG. 8 is the top view of the liquid dispenser.

FIG. 9 is the schematic drawing of the motherboard.

FIG. 10 is the front view of the electronic interface with the activation means.

The broken lines shown on these drawings do not form a part of the embodiments shown in the corresponding drawing(s).

### DETAILED DESCRIPTION OF CERTAIN INVENTIVE EMBODIMENTS

The detailed description set forth below in connection with the appended drawings is intended as a description of exemplary embodiments of the invention and is not intended to represent the only embodiments in which the invention may be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the invention.

The drawings presented herein are not to scale. Where dimensions are given, it is for illustrative purposes only and such dimensions shall in no way limit the invention disclosed herein. It is to be understood that different dimensions are contemplated for the dispensers according to embodiments.

As used herein, the term “Cloud” refers to servers that are accessed over the Internet, and the software and databases that run on those servers.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more”.

Embodiments of this invention relate to a liquid dispenser of handheld size suitable for personal use. The dispenser has multiple cartridges where different components, such as different liquids and/or liquid mixtures are stored. When used, mechanisms within each cartridge may be activated to dispense its internal content into a reservoir. The liquid then may be mixed together to create the final product consumable by a mammal, preferably a human being. The liquid may then be dispensed through a mouthpiece.

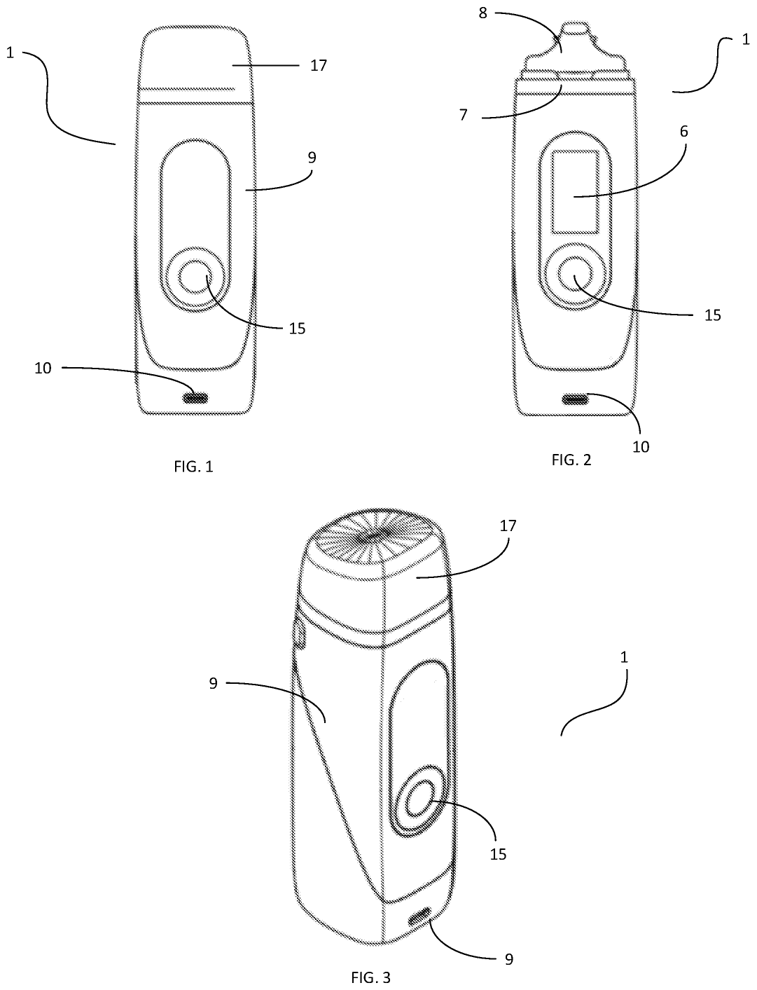

FIG. 1 illustrates the front view of the liquid dispenser 1 completely assembled. In this figure, the electronic interface is not shown. In embodiments, the liquid dispenser 1 may have an elongated body in generally cuboid shape with a circumference suitable for holding in a human being's hand. The liquid dispenser 1 may have rounded edges around the corners of the cuboid to prevent slipping from the hand. At the front side may be an activation means 15 where a user may press to activate the liquid dispenser 1. A USB gate 10 may be present on the front side, where the liquid dispenser 1 may be connected to an outside power source and/or an outside computing article. At the top may be a lid 17, which may be removable to expose the mouthpiece 8. The lid 17 may keep the mouthpiece 8 clean and prevent environmental contaminants. The lid 17 may be in a complementary shape to form a uniform body with other parts of the liquid dispenser 1 but may also be in other shapes suitable for its purpose. At the outside of the liquid dispenser 1 may be the housing 9, which may house components of the liquid dispenser 1.

For exemplary purposes, the liquid dispenser 1 may be of 100-200 mm in length and 30-60 mm in width. The bottom of the liquid dispenser 1 may be of substantially square shape with rounded corners and with approximately equal dimensions on four sides, however other shapes are contemplated, including substantially rectangular, rounded, oval, oblong, or other shapes.

FIG. 2 illustrates the front view of the liquid dispenser 1 in an embodiment of the present invention. An electronic interface 6 may be seen on the front side of the liquid dispenser 1. The electronic interface 6 may enable communication between the user and the liquid dispenser 1. It may also be configured to display certain operating parameters. The electronic interface 6 may show, for example, the amount of various liquids to be dispensed into the reservoir 3. Upon selection of these parameters, the liquid dispenser 1 may be activated to mix the mixture with the ratio as specified. For illustrative purposes, the screen may show the CBD liquid content to be dispensed is 1.5 ml, THC is 0.25 ml, and flavor is 1.25 ml. These amounts will be dispensed from their respective cartridge 2 into the reservoir 3 and mixed together before being consumed through the mouthpiece 8. Additional display on the electronic interface 6 may include the time, temperature, and battery indicator. The electronic interface 6 may also show other parameters, such as the history of dispensing. Additional parameters may be displayed. A user can navigate among the fields displayed on the electronic interface 6 using the activation means 15 to select options and commands to operate the liquid dispenser 1.

In FIG. 2, the lid 17 has been removed and the mouthpiece 8 may be seen at the top. The mouthpiece 8 may be constructed to enable the discharge of liquid in a constant stream and to enable dropping of liquid into another container. As shown in FIG. 2, the mouthpiece 8 is shaped similar to a funnel. Alternatively, the mouthpiece 8 may also be shaped with a long pipe that is retractable at a right angle. Other shapes and designs for the mouthpiece 8 suitable for use are contemplated. At the bottom of the mouthpiece 8 may be an RGB ring 7, which may be configured to emit light to indicate certain information concerning the liquid dispenser's 1 operation. Such information may be when the contents of the cartridges are being drawn up into the reservoir, when the contents of the cartridges are in the reservoir and ready to be dispensed, when the contents of the cartridges are being dispensed, low battery, empty cartridge(s), on/off animation, or successful pairing with Bluetooth, among other information. These parameters may alternatively be configured for display in an electronic interface.

In embodiments, the liquid dispenser 1 may comprise an activation means 15 to activate the liquid dispenser 1. A user can press on this activation means 15 to start the liquid dispenser's operation. The RGB ring 7 may be configured to light up to indicate that the liquid dispenser 1 has been activated and ready for operation.

In embodiments, the electronic interface 6 may be of 20-40 mm in length and 10-20 mm in width. As shown in FIG. 2, the electronic interface 6 is of rectangular shape but other shapes are contemplated. The activation means 15 may be of 5-10 mm in diameter. Other shapes and sizes for the activation means 15 are contemplated. The activation means 15 may be configured to have buttons on them to enable navigation of commands in the electronic interface 6. Buttons on the activation means are illustrated in FIG. 10.

FIG. 3 is a perspective view of the liquid dispenser 1 as shown in FIG. 1. The housing 9 may house others components inside. The housing 9 may be made of materials suitable to prevent breakage and slipping from the user's hands. Such materials may include durable plastic, stainless steel, ceramic, among other suitable materials. At the top of the liquid dispenser 1 may be a removable lid 17. The lid 17 may be of a shape and size to accommodate the mouthpiece 8, which the lid 17 covers. The lid 17 may also be of a shape and size that compliments the liquid dispenser 1 for aesthetic appeal.

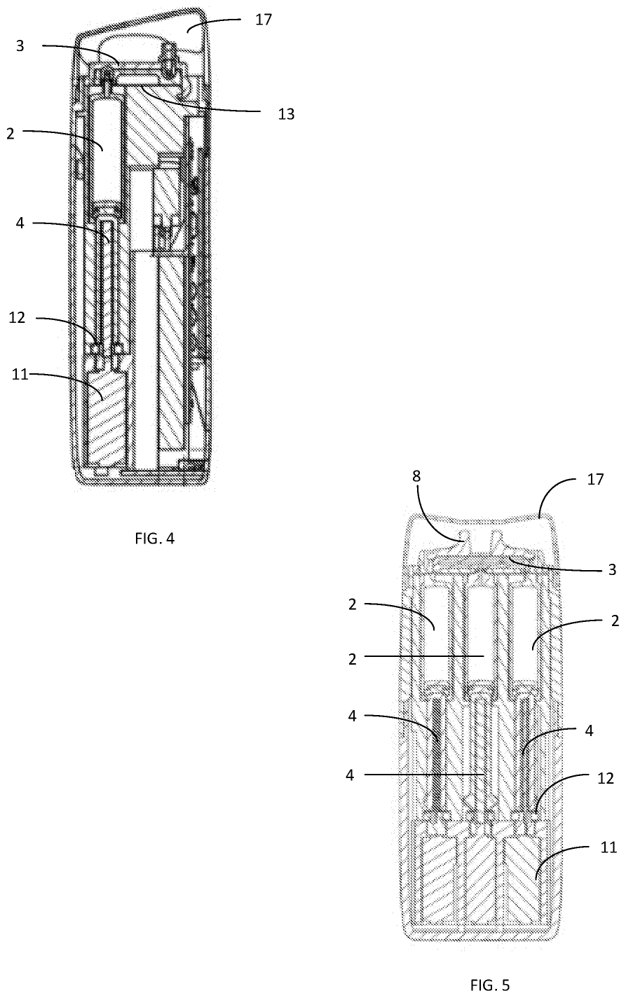

FIG. 4 is a side view of the liquid dispenser 1, showing a cartridge 2 wherein liquid may be stored, the cartridge 2 has been assembled into the liquid dispenser 1. The liquid may be premixed, containing at least one active ingredient mixed with other liquids and/or other ingredients to give the volume necessary for oral consumption. The liquid contained inside the cartridges may be a pure liquid, such as water or ethanol. The liquid may also be water-soluble powder dissolved in water. The liquid mixtures contained in the cartridges may only contain flavor and no active ingredients. Some liquid may be viscous oil extracted from plant sources such as oil containing cannabinoids. Other mixtures and kinds of liquid are contemplated.

In embodiments, the liquid dispenser 1 and the cartridges 2 may be constructed to prevent leakage and sustain strong shaking. The user may shake the liquid dispenser 1 to agitate the liquid mixtures inside the cartridges 2 and create an evenly distributed mixture. This may be desirable when the liquid mixture inside the cartridges 2 separate with time and form layers of suspension, thereby resulting in a mixture with uneven distribution of various components.

FIG. 5 shows a front of the liquid dispenser 1 with certain parts remove to show three cartridges as assembled into the liquid dispenser 1. For illustrative purposes, three cartridges are shown in FIG. 5, however the number of cartridges can be two or more, depending on the need of the user and the design. Each of the cartridges 2 may be shaped and sized to contain a liquid medicine or another liquid as needed for the mixture to be produced. Various cartridges with various contents may be made available such that a mixture of certain active ingredients may be produced in the end. In embodiments, a cartridge may be dedicated to storing flavors while another cartridge may be dedicated to storing preservatives, stabilizers, or emulsifiers, in addition to the active ingredient cartridge(s). A liquid in one cartridge may be pre-constituted to provide various ingredients, active or inactive. For example, a cartridge may have a formulation comprising cannabinoids and terpenes for flavoring, or other flavors.

As shown in FIG. 5, the cartridges 2 may be of piston shape, similar to that of a syringe to be used with an injection needle, to facilitate the dispensing means, which operates like a plunger in a syringe. The cartridges 2 may be made from materials capable of withstanding the plunging action. Compatibility of the cartridge's material with the liquid contained inside may be another consideration. The cartridge material may be a solid composite with advanced polymer material. Alternatively, where the liquid is a cannabinoid containing mixture, a durable glass cartridge may be preferred, especially when the mixture's viscosity may impede the movement of the dispensing means. Other liquid with high viscosity may also need this durable glass structure. Other durable materials for the cartridges are contemplated.

In embodiments, each cartridge 2 may have a dispensing means operatively connected to the cartridge 2. The dispensing means may move the liquid towards the cartridge outlet to dispense into the reservoir 3. As shown herein, the dispensing means comprises a pushing rod 4 similar to a piston pushing into the liquid while moving axially along the cartridge's body. Each pushing rod 4 in each dispensing means may be operated by an electronic motor 11 with a bearing 12 to constrain the movement of the pushing rod 4 to one direction, namely, to push the pushing rod 4 towards the cartridge outlet. Other mechanical mechanisms to move the liquid from the cartridges 2 into the reservoir 3 are contemplated.

In embodiments, at the top of each cartridge 2 may be a cartridge outlet, where liquid contained within the cartridge 2 may exit to enter the reservoir 3. The cartridge 2 therefore may physically and operatively connect with the reservoir 3 at the reservoir inlet, such that the cartridge outlet may line up with a reservoir inlet and liquid may pass through to enter the reservoir 3. The cartridge outlet may optionally be covered in a septum, which may open when the dispensing means 4 pushes the liquid out of the cartridge 2.

In embodiments, the reservoir 3 may comprise a hollow body with a space to contain liquid dispensed from various cartridges 2. The liquid dispensed from the various cartridges 2 may flow into this reservoir 3 through the cartridge outlet and the reservoir inlet. A reservoir holder 13 may be present at the bottom of the reservoir 3 and may physically and operatively connect to the housing 9 to provide structural support for the reservoir 3.

In embodiments, the reservoir 3 may have multiple reservoir inlets, which may operatively line up with the cartridge outlets, such that liquid may travel through the cartridge outlet and then through the reservoir inlet to enter the reservoir 3, where the liquid may be mixed with other liquids from other cartridges 2.

In embodiments, the mouthpiece 8 may sit on top of the reservoir 3, with an opening at the bottom connected to the reservoir 3. A septum may optionally be positioned near the opening of the mouthpiece 8 to prevent accidental dispensing of the liquid mixture from the reservoir 3. The mouthpiece 8 is where the liquid may travel through before reaching the user's mouth or dispensed into another container. The mouthpiece 8 may be sized and shaped to allow sucking on the mouthpiece 8 to consume the liquid dispensed. The mouthpiece 8 as shown herein may also be used to dispense liquid into another container by tipping the liquid dispenser 1 to point the mouthpiece 8 towards the other container.

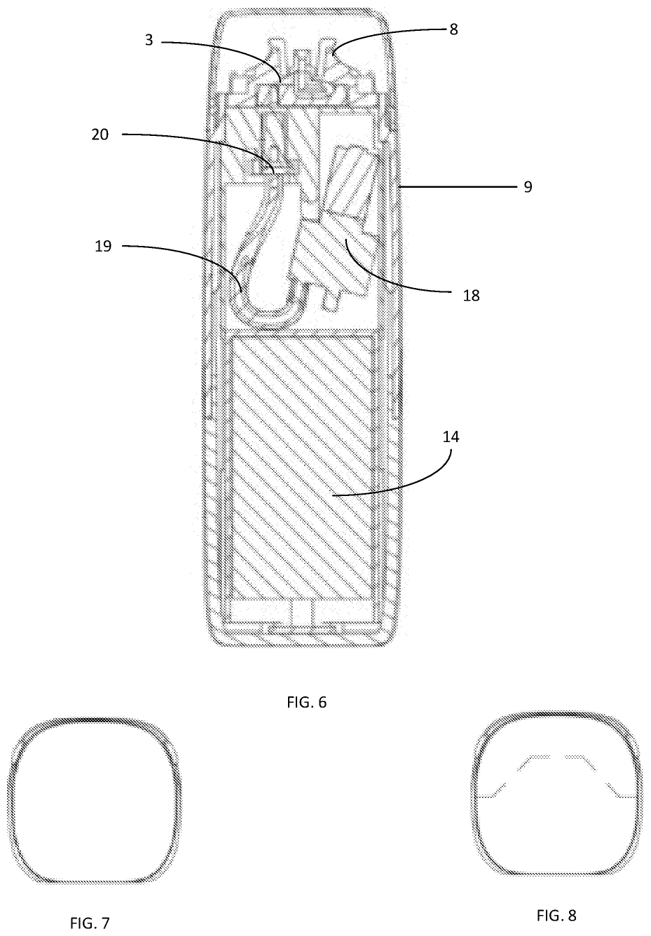

FIG. 6 shows a front view of the liquid dispenser 1 with the battery 14 and the pneumatic valve mechanism visible. These components may be assembled in front of the cartridges 2. In this view, the electronic interface 6 and the front components of the housing has been removed. The pneumatic valve 20 may sit between the reservoir 3 and the air channel 19. This pneumatic valve 20 may operate to push the finished liquid mixture in the reservoir 3 towards and out of the mouthpiece 8 to dispense the liquid mixture.

In embodiments, once the liquid mixture is ready, the dispensing may be started by command of the application and/or software, and the air pump 18 on the pneumatic valve mechanism may push air towards the reservoir 3 through the air channel 19, which may push liquid toward the mouthpiece 8, then the liquid may be dispensed out of the mouthpiece 8 for oral consumption. Alternatively, the user may also tilt the dispenser 1 and use it as a “dropper” to add the liquid dispensed from it into another body of fluid, for example, juice or coffee, for consumption. The pneumatic valve 20 may still need to be actuated to dispense the liquid from the liquid dispenser 1, even if the liquid dispenser 1 is tipped upside down. The mouthpiece 8 may be removable for easy cleaning and may be made of material that is stainless and durable to prevent breakage during removal and/or reassembling.

FIG. 7 shows the bottom view of the liquid dispenser 1. The bottom of the liquid dispenser 1 may be generally flat to allow the liquid dispenser 1 to stand on the bottom vertically, such that the liquid dispenser 1 can be stored in the same manner with bottles. As shown herein, the bottom of the dispenser 1 is of generally rectangular shape with rounded edges. Other shapes, such as oval, round, oblong, square, hexagon are contemplated. The flat surface at the bottom may allow the dispenser 1 to be stored upright regardless of the shape of the bottom.

FIG. 8 shows the top view of the lid 17, which may be of similar shape and size with the bottom. The lid 17 may be shaped and sized to accommodate the mouthpiece 8. As shown herein, the top of the lid 17 is of substantially rectangular shape with rounded edges. Other shapes, such as oval, round, oblong, square, hexagon are contemplated.

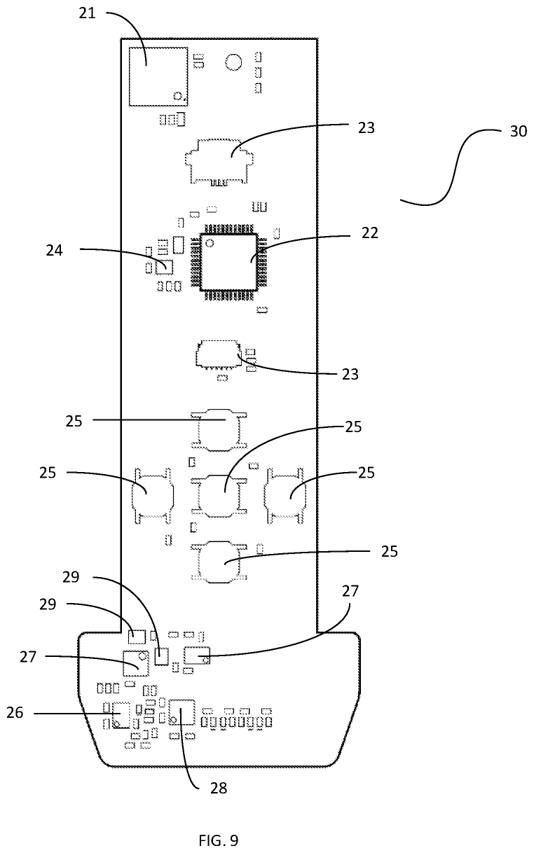

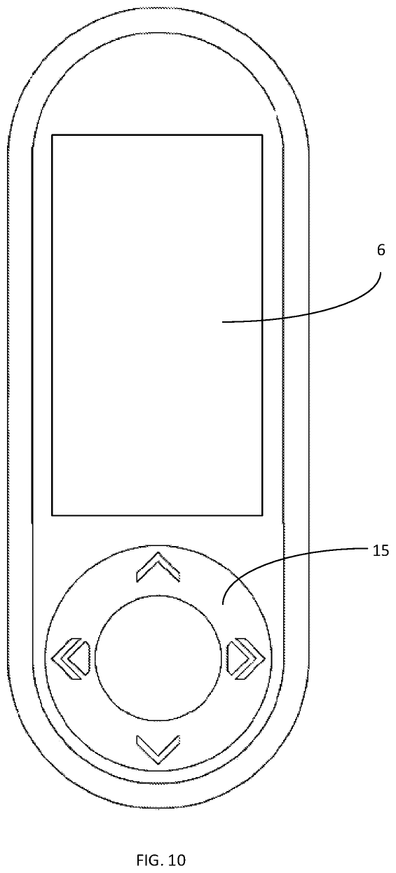

In embodiments, the liquid dispenser 1 may comprise a motherboard 30 to act as the electronic controller for the liquid dispenser 1. FIG. 9 is the schematic drawing of the motherboard 30 with different components illustrated by their functions. The motherboard 30 may be shaped to fit with the shape of the liquid dispenser 1 and it is illustrated in a T shape in FIG. 9. At the top of the motherboard 30 may be an antenna 21, which may be a Bluetooth Low Energy module or other types of antenna suitable for the purpose. A microcontroller 22 may be present, which in this case is a microcontroller with 128 kb embedded flash memory, for electronic control of the dispenser 1. Other processors may be used in place of the microcontroller. Connectors 23 may be present to connect the RGB ring 7 and/or the electronic interface 6 with the motherboard 30. Crystal oscillators 24 may be present to create correct frequency electrical signal for transmission. On the lower end of the motherboard 30 may be tactile switches 25, which are switches configured to interact with the electronic interface 6 when corresponding buttons are pressed. As shown in FIG. 9, there are five tactile switches, with the middle one acting like the “enter” key while the other four acting to move the active highlighted field on the electronic interface 6 up, down, left, or right.

At the bottom of the motherboard 30 may be components to control the use and charge of the battery 14, which may act as the power source. The motherboard 30 may have battery charge management chip 26, in particular a lithium ion battery charge management chip to manage charging time for the battery 14. The battery charge management chip may be a charge controller integrated circuit (IC). The motherboard 30 may also have a battery monitor 28 to monitor the battery charge level. Other components on the motherboard 30 may include voltage regulators 27 to regular voltage of the electrical currents received from the battery 14 and/or inductors 29. Other electrical components, such as capacitors, resistors, or printed circuitry may be present as needed.

The motherboard 30 may be assembled to the front of the battery 14 and to the back of the electronic interface 6. The motherboard 30 may also operatively interact with the electric motors 11 to operate the dispensing means and the air pump 18, which in turn may operate the pneumatic valve 20, as well as operating the electronic interface 6 and/or the RGB ring 7. Additionally, the motherboard may interact with remote computing processors to operate the liquid dispenser 1.

The motherboard is a printed circuit board where electronic components may be operationally connected and physically attached for mechanical stability. The motherboard may comprise a central processor, which may be a microcontroller, to collect signals and send commands to the various components of the liquid dispenser and may communicate with a remote computing article. An antenna may be present on the motherboard to collect signals from a remote computing processor and send signals to the same remote computing processor. Sensors may be present and connected with the electric motors to receive and transmit information concerning level of liquid in each cartridge. Connectors may be present on the motherboard to connect the motherboard with the RGB ring and/or other components. The components are connected by circuit board connectors present on the printed circuit board.

FIG. 10 is the front view of the electronic interface 6 with the activation means 15. The activation means 15 may be structured similar to a wheel with buttons corresponding to up, down, left, and right movement. In the middle of the activation means may be a fifth button acting like the “enter” key. The electronic interface 6 may display commands for a user to choose from using the buttons on the activation mechanism 15. Other information, such as remaining content of the cartridges, temperature, or time may also be displayed.

In embodiments, the electronic interface 6 may be assembled on top of the motherboard 30, facing outward for viewing and interaction by the user. The buttons on the activation means 15 may be lined up to correspond to the tactile buttons 25 on the motherboard 30, such that the tactile buttons 25 receive command when the buttons on the activation means 15 are pressed.

In embodiments, the components may be assembled together and fit into a housing 9. The housing 9 may provide structural support and shield the components inside. The housing 9 may be made of durable material that is electricity proof, such as durable plastic. Other materials for the housing 9 are contemplated. The housing 9 may comprise various components assembled together to form a complete structure surrounding other components of the liquid dispenser 1, except for the mouthpiece 8 and the lid 17.

A user may interact with the liquid dispenser 1 through the activation means 15 to choose different parameters on the electronic interface 6 to control the dispenser 1. A computer programming product, such as an application on a mobile computing device, such as a mobile phone or a tablet, or a software on a computing article, such as a desktop, laptop, or a computer embedded into another device, may interact with and control the dispenser 1. Information concerning the operation of the liquid dispenser 1 may be displayed on the electronic interface 6, the RGB ring 7, or on the user interface of the application and/or software on a remote computing article.

In embodiments, the liquid dispenser 1 may be configured to work at any angle of placement. Liquid may be dispensed when the liquid dispenser 1 is standing straight up, turned upside down, or tilted at any angle. The electronic interface 6 may change orientation as the liquid dispenser 1 is tilted to allow easy reading of information displayed.

In use of the invention, the liquid dispenser may be provided together with cartridges and with or without an application or software on a computing article to control and operate the liquid dispenser. The cartridges may be prefilled with active ingredients and/or flavor in liquid form, the quantity and components of which may be stored in an electronic information storage means, which may be Near Field Communication (NFC) tags or Quick Response (QR) codes. This information may be read by the application or software controlling the liquid dispenser.

A user may activate the liquid dispenser by pressing the activation means. Once powered up, the dispenser may communicate with the computer programming product on a remote computing device. The computer programming product may interact with the user to receive certain operation parameters, such as which liquid, how much to dispense, or mixing time. The application or software may then activate the liquid dispenser and cause the various cartridges to dispense their content according to the received parameters. The application or software may also have pre-built capability to receive a dosage specification and determine mixing parameters and quantities to prepare the mixture. Alternatively, a person using a remote computing article having an application or software configured to communicate with the liquid dispenser may also determine operating parameters and cause the liquid dispenser to operate with such parameters. Lastly, the user may use the buttons on the activation means to navigate through the menu on the electronic interface on the liquid dispenser to specify operation parameters for the liquid dispenser.

A user may also use the buttons on the activation means to select operating parameters for the liquid dispenser. Using the up, down, left, and right buttons, the user can choose various different parameters by navigating through a menu. Once the parameters are set, the user can order the dispenser to start mixing and dispensing the liquid. A dispenser may be configured such that it can be operated by either way of control.

The signal communicated from the application or software may be received by an electronic controller, namely, an electrical circuit board or motherboard configured to electronically operate the dispenser. Alternatively, the information entered using the electronic interface may also be received by the motherboard. The motherboard may then activate the dispensing mechanism to move and dispense the specified amount from each cartridge. Liquid may move from each cartridge and enter the reservoir to form a liquid mixture.

In embodiments, once mixing is completed, the liquid mixture may be dispensed for use. Upon receiving the command from the electronic controller, the air pump may activate and push air through the air channel to the pneumatic valve and open it. Liquid may flow from the reservoir into the mouthpiece and reach the user's mouth or is dispensed into another liquid or container.

In embodiments, the application or software used to control the liquid dispenser may interact with users to receive specified operating parameters, including, but not limited to, the amount to dispense from each cartridge. Information retrieved from the electronic information storage means may be used to decide on the specified operating parameters. The application or software may also have pre-built operating protocols that may be activated upon receiving information from the electronic information means, which may control the operating parameters of the liquid dispenser. A remote computing article with a corresponding application and/or software and controlled by another person other than the user may also send command to the liquid dispenser to dictate operating parameters.

Each cartridge may carry an NFC tag or QR code in the form of a chip, tag, or sticker with information concerning the content as well as the user. Information on the content may be active ingredient(s), amount, producer, used by date, or prescriber, among other information. When the cartridge is filled for the user with a specific prescription, the information included in the NFC tag or QR code may also include the user's uniquely identifying information to track the user's progress in medicine consumption. Such information may be stored in a remote computing article or in the Cloud and used by medical care providers to make care decision for the individual.

Electronic control of the dispenser may be through electronic components centered on a motherboard powered by a battery, which may be rechargeable. The motherboard may be placed on the front of the battery and be powered by the battery. The motherboard may comprise an antenna, a microcontroller or another kind of processor, connectors, and tactile switches, among other necessary components. The components of the motherboard may be connected by printed circuitry.

Communication between the dispenser and the computer programming product, which may be an application or software, on a remote computing article may be through wireless communication using an antenna, such as a Bluetooth Low Energy module, on the motherboard. Signals from the remote computing article generated by the computer programming product may be received by an antenna, which is a component on the motherboard within the dispenser. The application or software many generate commands based on input by a user interacting with the application or software, or by programmed commands.

In embodiments, the battery in the liquid dispenser may be charged through a USB connection, which may be operatively and physically connected to a USB board. Information may be communicated from the application or the software to the liquid dispenser through the USB connection, even though this is a rare occurrence. This communication may be in addition to or in lieu of wireless communication.

During operation, information from the dispenser may be collected and communicated to the application or software and stored in a computing article or in the Cloud. The dispenser may collect and communicate information concerning which kind of cartridge is used, the content and active ingredients used in each cartridge, the producer of each cartridge, the amount used, the frequency of use, geolocation of the usage, among other parameters. For each user, this information may be stored and tracked to provide to medical care providers to make care decisions. Information from various liquid dispensers may be collected and aggregated for research purposes.

The parts herein may be made by methods known in the art, such as injection molding or relatively precise machining. The housing may be made from plastic with heat tolerance or stainless steel and may be electrically separated from the rest of the components and/or electricity proof. The motherboard may be made from a printed circuit board with electronic components physically and electrically connected to the connections present on the printed circuit board. Suitable materials may be selected to suit the purpose of each components within the dispenser.

All references, including publications, patent applications, and patents cited herein are hereby incorporated by reference to the same extent as if each reference were individually and specifically indicated to be incorporated by reference and were set forth in its entirety herein.

It will be readily apparent to those skilled in the art that a number of modifications and changes may be made without departing from the spirit and the scope of the present invention. It is to be understood that any ranges, ratios, and range of ratios that can be derived from any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art will appreciate that such values are unambiguously derivative from the data presented herein.

What is claimed is:

1. A system for mixing and dispensing liquid, comprising:
a reservoir configured to receive liquid from at least two sources, the reservoir having inlets capable of receiving liquid;
at least two cartridges, each of the cartridge comprises:
a dispensing means within the cartridge to dispense a liquid from each cartridge into a mixing reservoir;
space within the cartridge to store a volume of liquid; and
an outlet at one end of the cartridge;
a pneumatic pressure valve operatively connected to the reservoir;
an electronic controller embedded within the system and configured to operate the system;
an electronic interface present on the system and configured to interact with a user to operate the system;
a power source;
a mouthpiece with an opening connected to the reservoir; and
a housing to house the above components,
wherein each of the cartridges stores a different liquid mixture and connects physically and operatively to the reservoir at one of the inlets.

2. The system of claim 1, wherein at least one of the liquid mixtures comprises at least one cannabinoid.

3. The system of claim 1, wherein at least of one of the liquid mixtures is viscous at room temperature.

4. The system of claim 1, wherein at least one of the liquid mixtures comprises a flavor.

5. The system of claim 1, wherein the outlets are covered with a mechanical septum.

6. The system of claim 1, wherein the liquid cartridges are pre-made and replaceable.

7. The system of claim 1, wherein each of the liquid cartridges further comprises information contained in an electronic information storage means.

8. The system of claim 7, wherein the electronic information storage means is Near Field Communication or Quick Response code.

9. The system of claim 1, further comprising an activation means.

10. The system of claim 1, further comprising means to wirelessly communicate with other processors embedded within the electronic controller.

11. The system of claim 10, further comprising a computer programming product configured to wirelessly communicate with and send orders to the electronic controller.

12. The system of claim 11, wherein the computer programming product is an application or a software capable of operating on a computing article and configured to interact with a user at a user interface displayed on the computing article.

13. The system of claim 12, wherein the computing article is a mobile computing device, a desktop, a laptop, or a computer embedded into another electronic device.

14. The system of claim 13, wherein the computer programming product is further configured to collect information from the electronic information storage means present on the cartridge.

15. The system of claim 14, wherein the computer programming product is configured to control operating parameters of the system.

16. The system of claim 15, wherein the operating parameters are dispensing amount from each cartridge, mixture ratio, time of consumption, frequency of consumption, and place of consumption.

17. The system of claim 16, wherein the computer programming product is further configured to collect information on operating parameters from the system and transmit collected information to other computing articles.

18. The system of claim 17, wherein collected information is stored on a computing article, aggregated, and analyzed to optimize the system's operation or to conduct medical research.

19. A method to provide a liquid mixture to a user, comprising:
providing to a user the system of claim 17;
assembling pre-filled cartridges into the system;
activating the dispenser using the activation means;
control operating parameters using the computer programming product on the remote computing article;
waiting for the system to prepare the liquid mixture; and
consuming the liquid mixture orally.

20. A method to conduct medical studies, comprising the steps of:
providing to a user the system of claim 18;
opening the computer programming product on the remote computing processor;
setting operating parameters for the system;
starting the operating procedure for the system to provide the liquid mixture for the user's consumption;
collecting data from the system operation;
transmitting collected data to a remote computing processor;
storing collected data in a computing article;
repeating the above steps with various systems and various users for a finite time;
analyzing stored data to make medical care decision; and
aggregating data from different users and conducting research and study based on the aggregated data.
